(19) United States
(12) Patent Application Publication
(10) Pub. No.: US 2006/0183765 A1
(43) Pub. Date: Aug. 17, 2006

(54) Methods of treating cancer using an FPT inhibitor and antineoplastic agents

(76) Inventors: David L. Cutler, Moorestown, NJ (US); Michael L. Meyers, Hastings-on-Hudson, NY (US); Charles Baum, Westfield, NJ (US); Sara L. Zaknoen, Hoboken, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/396,892

(22) Filed: Apr. 3, 2006

Related U.S. Application Data
(62) Division of application No. 10/303,259, filed on Nov. 25, 2002.
(60) Provisional application No. 60/334,411, filed on Nov. 30, 2001.

(51) Int. Cl. A61K 31/473; A61K 31/704; A61K 31/454; A61K 31/4745; A61K 31/28; A61K 33/24

(52) U.S. Cl. 514/290; 514/449; 514/283; 514/34; 514/365; 514/323; 424/649; 514/492

(57) ABSTRACT

Disclosed is a method of treating cancer in a patient in need of such treatment comprising administering a therapeutically effective amount of an FPT inhibitor and therapeutically effective amounts of one or more antineoplastic agents. Methods of treating non small cell lung cancer, CML, AML, non-Hodgkin's lymphoma and multiple myeloma are disclosed.

### REFERENCE TO RELATED APPLICATION

This application claims the benefit of Provisional Application Ser. No. 60/334411 filed on Nov. 30, 2001.

### BACKGROUND

WO 98/54966 published Dec. 10, 1998 discloses methods of treating cancer by administering at least two therapeutic agents selected from a group consisting of a compound which is an antineoplastic agent and a compound which is an inhibitor of prenyl-protein transferase (e.g., a farnesyl protein transferase inhibitor).

Farnesyl Protein Transferase (FPT) Inhibitors are known in the art, see for example U.S. Pat. No. 5,874,442 issued Feb. 23, 1999. Methods of treating proliferative diseases (e.g., cancers) by administering an FPT inhibitor in conjunction with an antineoplastic agent and/or radiation therapy are also known, see for example U.S. Pat. No. 6,096,757 issued Aug. 1, 2000.

Shih et al., “The farnesyl protein transferase inhibitor SCH66336 synergizes with taxanes in vitro and enhances their antitumor activity in vivo”, Cancer Chemother. Pharmacol. (2000) 46: 387-393 discloses a study of the combination of SCH 66336 with paclitaxel, and SCH 66336 with docetaxel on certain cancer cell lines.

WO 01/45740 published Jun. 28, 2001 discloses a method of treating cancer (breast cancer) comprising administering a selective estrogen receptor modulator (SERM) and at least one farnesyl transferase inhibitor (FTI). FTI-277 is the exemplified FTI.

The WEB site http://www.osip.com/press/pr/07-25-01 discloses a press release of OSI Pharmaceuticals. The press release announces the initiation of a Phase III clinical trial evaluating the use of the epidermal growth factor inhibitor Tarceva (TM) (OSI-774) in combination with Carboplatin (Paraplatin®) and Paclitaxel (Taxol®) for the treatment of Non Small Cell Lung Cancer.

The WEB site http://cancertrials.nci.nih.gov/types/lung/iressal12100.html in a disclosure posted Dec. 14, 2000 discloses the following list of open clinical trials for advanced (stage IIIB and IV) non-small cell lung cancer, from NCI's clinical trials database:
(1) phase III Randomized Study of ZD 1839 (IRESSA, an epidermal growth factor inhibitor) combined with gemcitabine and cisplatin in chemotherapy-naïve patients with Stage IIIB or IV. non-small cell lung cancer; and

(2) phase III Randomized Study of ZD 1839 (IRESSA, an epidermal growth factor inhibitor) combined with paclitaxel and carboplatin in chemotherapy-naïve patients with Stage IIIB or IV non-small cell lung cancer.

WO 01/56552 published Aug. 9, 2001 discloses the use of an FPT inhibitor for the preparation of a pharmaceutical composition for treating advanced breast cancer. The FPT inhibitor may be used in combination with one or more other treatments for advanced breast cancer especially endocrine therapy such as an antiestrogen agent such as an estrogen receptor antagonist (e.g., tamoxifen) or a selective estrogen receptor modulator or an aromatase inhibitor. Other anti-cancer agents which may be employed include, amongst others, platinum coordination compounds (such as cisplatin or carboplatin), taxanes (such as paclitaxel or docetaxel), anti-tumor nucleoside derivatives (such as gemcitabine), and HER2 antibodies (such as trastzumab).

WO 01/62234 published Aug. 30, 2001 discloses a method of treatment and dosing regimen for treating mammalian tumors by the discontinuous administration of a farnesyl transferase inhibitor over an abbreviated one to five day dosing schedule. Disclosed is a regimen wherein the farnesyl protein transferase inhibitor is administered over a one to five day period followed by at least two weeks without treatment. It is disclosed that in previous studies farnesyl protein transferase inhibitors have been shown to inhibit the growth of mammalian tumors when administered as a twice daily dosing schedule. It is further disclosed that the administration of a farnesyl protein transferase inhibitor in a single dose daily for one to five days produced a marked suppression of tumor growth lasting one to at least 21 days. It is also disclosed that the FTI may be used in combination with one or more other anti-cancer agents such as, platinum coordination compounds (e.g., cisplatin or carboplatin), taxane compounds (e.g., paclitaxel or docetaxel), anti-tumor nucleoside derivatives (e.g., gemcitabine), HER2 antibodies (e.g., trastzumab), and estrogen receptor antagonists or selective estrogen receptor modulators (e.g., tamoxifen).

WO 01/64199 published Sep. 7, 2001 discloses a combination of particular FPT inhibitors with taxane compounds (e.g., paclitaxel or docetaxel) useful in the treatment of cancer.

Those skilled in the art have a continued interest in finding specific combinations of compounds that would provide more effective cancer treatments. A welcome contribution to the art would be a method of treating cancer using specific combinations of compounds that results in increased survival rates of patients with cancer. This invention provides such a contribution.

### SUMMARY OF THE INVENTION

This invention provides a method of treating cancer in a patient in need of such treatment comprising administering a therapeutically effective amount of an FPT inhibitor and therapeutically effective amounts of at least two different antineoplastic. agents selected from the group consisting of: (1) taxanes, (2) platinum coordinator compounds, (3) epidermal growth factor (EGF) inhibitors that are antibodies, (4) EGF inhibitors that are small molecules, (5) vascular endolithial growth factor (VEGF) inhibitors that are antibodies, (6) VEGF kinase inhibitors that are small molecules, (7) estrogen receptor antagonists or selective estrogen receptor modulators (SERMs), (8) anti-tumor nucleoside derivatives, (9) epothilones, (10) topoisomerase inhibitors, (11) vinca alkaloids, (12) antibodies that are inhibitors of αVβ3 integrins; (13) small molecules that are inhibitors of αVβ3 integrins; (14) folate antagonists; (15) ribonucleotide reductase inhibitors; (16) anthracyclines; (17) biologics; (18) thalidomide (or related imid); and (19) Gleevec.

This invention also provides a method of treating cancer in a patient in need of such treatment comprising administering therapeutically effective amounts of an FPT inhibitor and an antineoplastic agent selected from the group consisting of: (1) EGF inhibitors that are antibodies, (2) EGF inhibitors that are small molecules, (3) VEGF inhibitors that are antibodies, and (4) VEGF inhibitors that are small molecules. Radiation therapy can also be used in conjunction with the above combination therapy, i.e., the above method using a combination of FPT inhibitor and antineoplastic agent can also comprise the administration of a therapeutically effect amount of radiation.

This invention also provides a method of treating leukemias (e.g., acute myeloid leukemia (AML), and chronic myeloid leukemia (CML)) in a patient in need of such treatment comprising administering therapeutically effective amounts of an FPT inhibitor and: (1) Gleevec and interferon to treat CML; (2) Gleevec and pegylated interferon to treat CML; (3) an anti-tumor nucleoside derivative (e.g., Ara-C) to treat AML; or (4) an anti-tumor nucleoside derivative (e.g., Ara-C) in combination with an anthracycline to treat AML.

This invention also provides a method of treating non-Hodgkin's lymphoma in a patient in need of suclitreatment comprising administering therapeutically effective amounts of an FPT inhibitor and: (1) a biologic (e.g., Rituxan); (2) a biologic (e.g., Rituxan) and an anti-tumor nucleoside derivative (e.g., Fludarabine); or (3) Genasense (antisense to BCL-2).

This invention also provides a method of treating multiple myeloma in a patient in need of such treatment comprising administering therapeutically effective amounts of an FPT inhibitor and: (1) a proteosome inhibitor (e.g., PS-341 from Millenium); or (2) Thalidomide (or related imid).

### DETAILED DESCRIPTION OF THE INVENTION

As used herein, unless indicated otherwise, the term “AUC” means “Area Under the Curve”.

As used herein, unless indicated otherwise, the term “effective amount” means a therapeutically effective amount. For example, the amount of the compound (or drug), or radiation, that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor. For example, in the treatment of lung cancer (e.g., non small cell lung cancer) a therapeutically effective amount is that amount that alleviates or eliminates cough, shortness of breath and/or pain. Also, for example, a therapeutically effective amount of the FPT inhibitor is that amount which results in the reduction of farnesylation. The reduction in farnesylation may be determined by the analysis of pharmacodynamic markers such as Prelamin A and HDJ-2 (DNAJ-2) using techniques well known in the art.

As used herein, unless indicated otherwise, the term “different” as used in the phrase “different antineoplastic agents” means that the agents are not the same compound or structure. Preferably, “different” as used in the phrase “different antineoplastic agents” means not from the same class of antineoplastic agents. For example, one antineoplastic agent is a taxane, and another antineoplastic agent is a platinum coordinator compound.

As used herein, unless indicated otherwise, the term “compound” with reference to the antineoplastic agents includes the agents that are antibodies.

As used herein, unless indicated otherwise, the term “consecutively” means one following the other.

As used herein, unless indicated otherwise, the term “concurrently” means at the same time.

As described herein, unless otherwise indicated, the use of a drug or compound in a specified period (e.g., once a week, or once every three weeks, etc.) is per treatment cycle.

The methods of this invention are directed to the use of a combination of drugs (compounds) for the treatment of cancer, i.e., this invention is directed to a combination therapy for the treatment of cancer. Those skilled in the art will appreciate that the drugs are generally administered individually as a pharmaceutical composition. The use of a pharmaceutical composition comprising more than one drug is within the scope of this invention.

The antineoplastic agents are usually administered in the dosage forms that are readily available to the skilled clinician, and are generally administered in their normally prescribed amounts (as for example, the amounts described in the Physician's Desk Reference, 55^{th} Edition, 2001, or the amounts described in the manufacture's literature for the use of the agent).

For example, the FPT inhibitor can be administered orally as a capsule, and the antineoplastic agents can be administered intravenously, usually as an IV solution. The use of a pharmaceutical composition comprising more than one drug is within the scope of this invention.

The FPT inhibitor used in this invention is the compound: 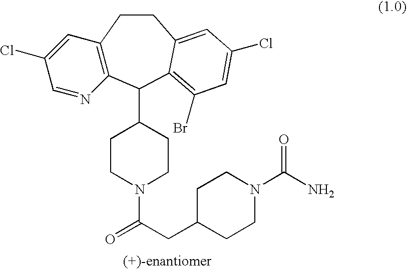
which compound can also be represented by the formula: 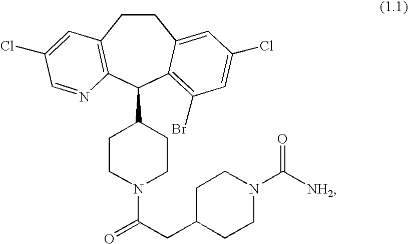
that is ((11R) 4[2[4-(3,10-dibromo-8-chloro-6,11-dihydro-5H-benzo[5,6]cyclohepta[1,2-b]pyridin-11yl)-1-piperazinyl]-2-oxoethyl]-1-piperidinecarboxamide)). This compound is described in U.S. Pat. No. 5,874,442 issued Feb. 23, 1999, and WO99/32118 published Jul. 1, 1999, the disclosures of which are incorporated herein by reference thereto.

This invention provides a method of treating cancer comprising administering to a patient in need of such treatment therapeutically effective amounts of:
(a) the FPT inhibitor 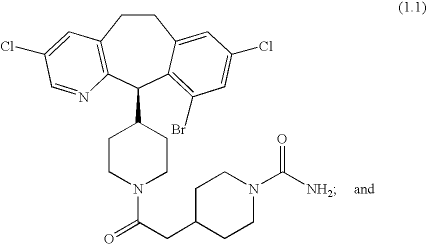
(b) at least two different antineoplastic agents selected from the group consisting of:
   (1) taxanes;
   (2) platinum coordinator compounds;
   (3) EGF inhibitors that are antibodies;
   (4) EGF inhibitors that are small molecules;
   (5) VEGF inhibitors that are antibodies;
   (6) VEGF kinase inhibitors that are small molecules;
   (7) estrogen receptor antagonists or selective estrogen receptor modulators;
   (8) anti-tumor nucleoside derivatives;
   (9) epothilones;
   (10) topoisomerase inhibitors;
   (11) vinca alkaloids;
   (12) antibodies that are inhibitors of αVβ3 integrins;
   (13) small molecule inhibitors of αVβ3 integrins;
   (14) folate antagonists;
   (15) ribonucleotide reductase inhibitors;
   (16) anthracyclines;
   (17) biologics;
   (18) Thalidomide (or related Imid); and
   (19) Gleevec.

This invention also provides a method of treating cancer comprising administering to a patient in need of such treatment therapeutically effective amounts of:
(a) the FPT inhibitor 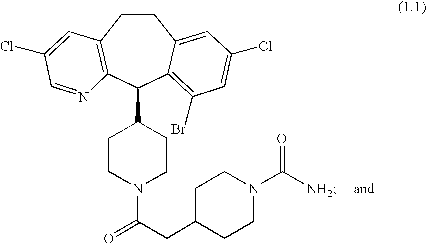
(b) at least two different antineoplastic agents selected from the group consisting of:
   (1) taxanes;
   (2) platinum coordinator compounds;
   (3) EGF inhibitors that are antibodies;
   (4) EGF inhibitors that are small molecules;
   (5) VEGF inhibitors that are antibodies;
   (6) VEGF kinase inhibitors that are small molecules;
   (7) estrogen receptor antagonists or selective estrogen receptor modulators;
   (8) anti-tumor nucleoside derivatives;
   (9) epothilones;
   (10) topoisomerase inhibitors;
   (11) vinca alkaloids;
   (12) antibodies that are inhibitors of αVβ3 integrins;
   (13) small molecule inhibitors of αVβ3 integrins; and
   (14) folate antagonists.
   (15) ribonucleotide reductase inhibitors;
   (16) anthracyclines;
   (17) biologics; and
   (18) Thalidomide (or related Imid).

This invention also provides a method of treating cancer comprising administering to a patient in need of such treatment therapeutically effective amounts of:
(a) the FPT inhibitor 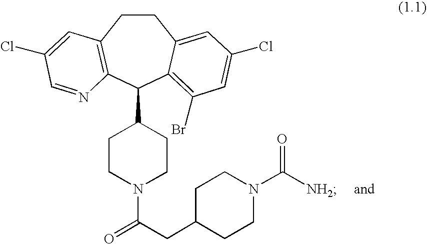
(b) at least two different antineoplastic agents selected from the group consisting of:
   (1) taxanes;
   (2) platinum coordinator compounds;
   (3) EGF inhibitors that are antibodies;
   (4) EGF inhibitors that are small molecules;
   (5) VEGF inhibitors that are antibodies;
   (6) VEGF kinase inhibitors that are small molecules;
   (7) estrogen receptor antagonists or selective estrogen receptor. modulators;
   (8) anti-tumor nucleoside derivatives;
   (9) epothilones;
   (10) topoisomerase inhibitors;
   (11) vinca alkaloids;
   (12) antibodies that are inhibitors of αVβ3 integrins;
   (13) small molecule inhibitors of αVβ3 integrins; and
   (14) folate antagonists.
   (15) ribonucleotide reductase inhibitors;
   (16) anthracyclines; and
   (17) biologics

This invention also provides a method of treating cancer comprising administering to a patient in need of such treatment therapeutically effective amounts of:
(a) the FPT inhibitor 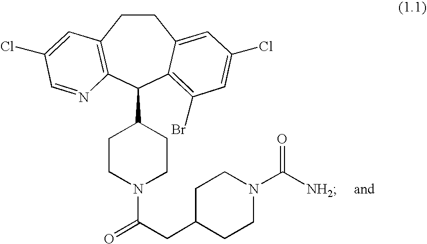
(b) at least two different antineoplastic agents selected from the group consisting of:
   (1) taxanes;
   (2) platinum coordinator compounds;
   (3) EGF inhibitors that are antibodies;
   (4) EGF inhibitors that are small molecules;
   (5) VEGF inhibitors that are antibodies;
   (6) VEGF kinase inhibitors that are small molecules;
   (7) estrogen receptor antagonists or selective estrogen receptor modulators;
   (8) anti-tumor nucleoside derivatives;
   (9) epothilones;
   (10) topoisomerase inhibitors;
   (11) vinca alkaloids;
   (12) antibodies that are inhibitors of αVβ3 integrins; and
   (13) small molecule inhibitors of αVβ3 integrins.

This invention also provides a method of treating non small cell lung cancer comprising administering to a patient in need of such treatment therapeutically effective amounts of:
(a) the FPT inhibitor 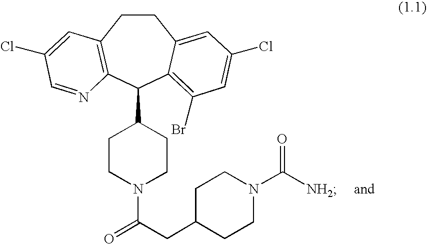
(b) at least two different antineoplastic agents selected from the group consisting of:
   (1)taxanes;
   (2) platinum coordinator compounds;
   (3) EGF inhibitors that are antibodies;
   (4) EGF inhibitors that are small molecules;
   (5) VEGF inhibitors that are antibodies;
   (6) VEGF kinase inhibitors that are small molecules;
   (7) estrogen receptor antagonists or selective estrogen receptor modulators;
   (8) anti-tumor nucleoside derivatives;
   (9) epothilones;
   (10) topoisomerase inhibitors;
   (11) vinca alkaloids;
   (12) antibodies that are inhibitors of αVβ3 integrins; and
   (13) small molecule inhibitors of αVβ3 integrins.

This invention also provides a method of treating non small cell lung cancer comprising administering to a patient in need of such treatment therapeutically effective amounts of:
(a) the FPT inhibitor 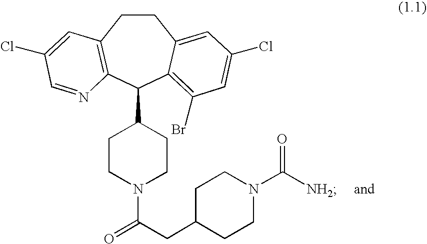
(b) at least two different antineoplastic agents selected from the group consisting of:
   (1) taxanes;
   (2) platinum coordinator compounds;
   (3) anti-tumor nucleoside derivatives;
   (4) topoisomerase inhibitors; and
   (5) vinca alkaloids.

This invention also provides a method of treating non small cell lung cancer in a patient in need of such treatment comprising administering therapeutically effective amounts of:
(a) an FPT inhibitor of the formula: 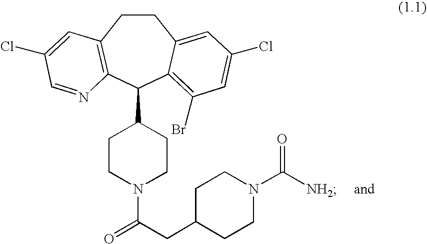
(b) Carboplatin; and
(c) Paclitaxel.

This invention also provides a method of treating non small cell lung cancer in a patient in need of such treatment comprising administering therapeutically effective amounts of:
(a) an FPT inhibitor of the formula: 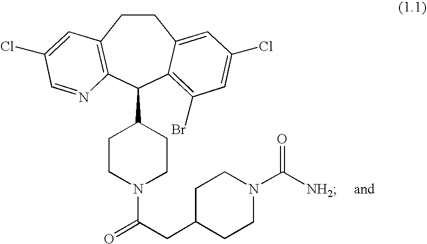
(b) Cisplatin; and
(c) Gemcitabine.

This invention also provides a method of treating non small cell lung cancer in a patient in need of such treatment comprising administering therapeutically effective amounts of:
(a) an FPT inhibitor of the formula: 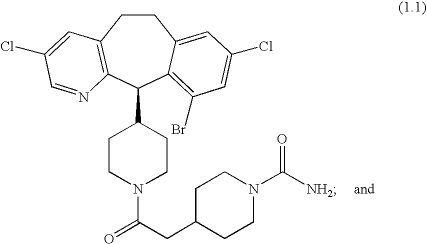
(b) Carboplatin; and
(c) Docetaxel.

This invention also provides a method of treating non small cell lung. cancer in a patient in need of such treatment comprising administering therapeutically effective amounts of:
(a) an FPT inhibitor of the formula: 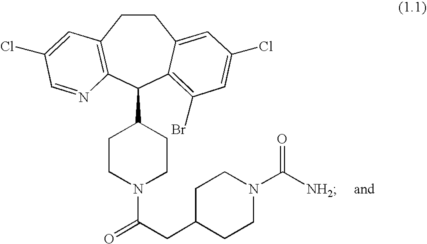
(b) Carboplatin; and
(c) Gemcitabine.

This invention also provides a method of treating cancer in a patient in need of such treatment comprising administering therapeutically effective amounts of:
(a) an FPT inhibitor of the formula: 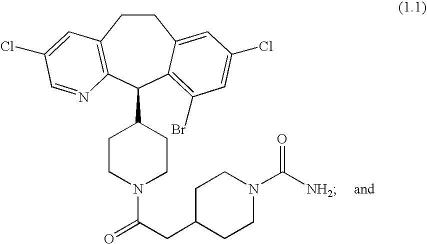
(b) an antineoplastic agent selected from the group consisting of:
   (1) EGF inhibitors that are antibodies;
   (2) EGF inhibitors that are small molecules;
   (3) VEGF inhibitors that are antibodies; and
   (4) VEGF kinase inhibitors that are small molecules.

This invention also provides a method of treating squamous cell cancer of the head and neck in a patient in need of such treatment comprising administering therapeutically effective amounts of:
(a) an FPT inhibitor of the formula: 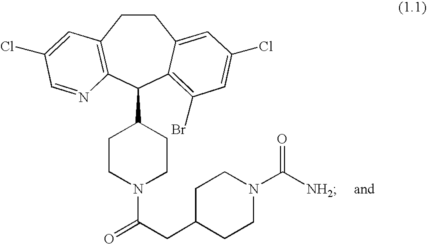
(b) one or more antineoplastic agents selected from the group consisting of:
   (1) taxanes; and
   (2) platinum coordinator compounds.

This invention also provides a method of treating squamous cell cancer of the head and neck in a patient in need of such treatment comprising administering therapeutically effective amounts of:
(a) an FPT inhibitor of the formula: 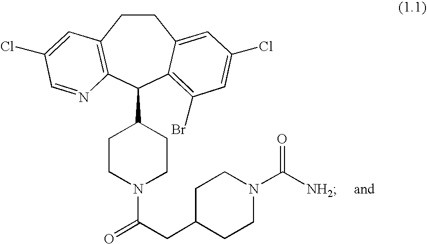
(b) at least two different antineoplastic agents selected from the group consisting of:
   (1) taxanes;
   (2) platinum coordinator compounds; and
   (3) anti-tumor nucleoside derivatives (e.g., 5-Fluorouracil).

This invention also provides a method of treating CML in a patient in need of such treatment comprising administering therapeutically effective amounts of:
(a) an FPT inhibitor of the formula: 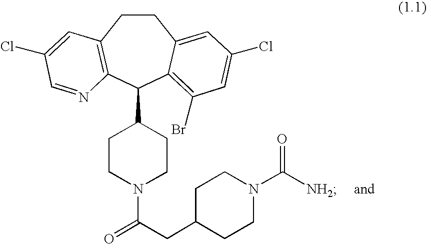
(b) Gleevec; and
(c) interferon (e.g., Intron-A).

This invention also provides a method of treating CML in a patient in need of such treatment comprising administering therapeutically effective amounts of:
(a) an FPT inhibitor of the formula: 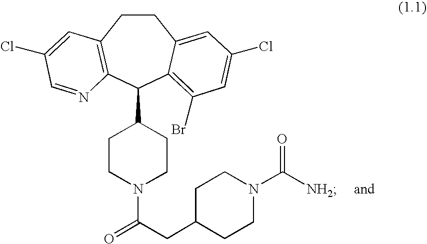
(b) Gleevec; and
(c) pegylated interferon (e.g., Peg-Intron, and Pegasys).

This invention also provides a method of treating AML in a patient in need of such treatment comprising administering therapeutically effective amounts of:
(a) an FPT inhibitor of the formula: 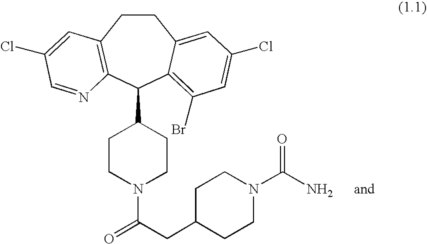
(b) an anti-tumor nucleoside derivative (e.g., Cytarabine (i.e., Ara-C)).

This invention also provides a method of treating AML in a patient in need of such treatment comprising administering therapeutically effective amounts of:
(a) an FPT inhibitor of the formula: 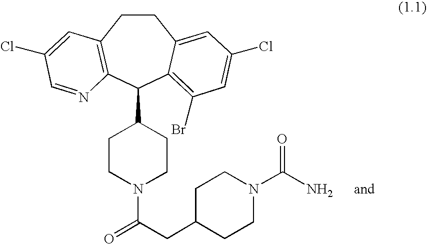
(b) an anti-tumor nucleoside derivative (e.g., Cytarabine (i.e., Ara-C)); and
(c) an anthracycline.

This invention also provides a method of treating non-Hodgkin's lymphoma in a patient in need of such treatment comprising administering therapeutically effective amounts of:
(a) an FPT inhibitor of the formula: 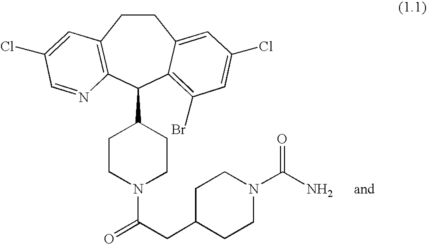
(b) Rituximab (Rituxan).

This invention also provides a method of treating non-Hodgkin's lymphoma in a patient in need of such treatment comprising administering therapeutically effective amounts of:
(a) an FPT inhibitor of the formula: 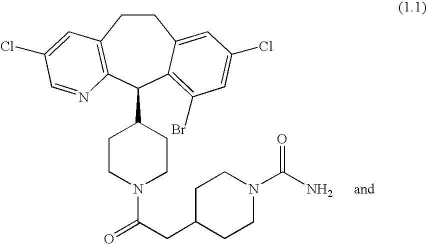
(b) Rituximab (Rituxan); and
(c) an anti-tumor nucleoside derivative (e.g., Fludarabine (i.e., F-ara-A).

This invention also provides a method of treating non-Hodgkin's lymphoma in a patient in need of such treatment comprising administering therapeutically effective amounts of:
(a) an FPT inhibitor of the formula: 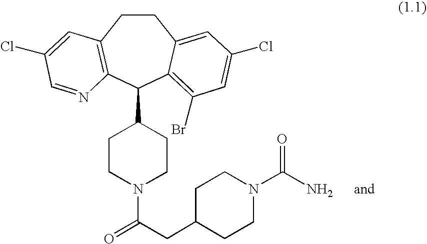
(b) Genasense (antisense to BCL-2).

This invention also provides a method of treating multiple myeloma in a patient in need of such treatment comprising administering therapeutically effective amounts of:
(a) an FPT inhibitor of the formula: 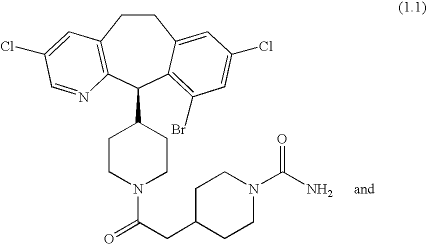
(b) a proteosome inhibitor (e.g., PS-341 (Millenium)).

This invention also provides a method of treating multiple myeloma in a patient in need of such treatment comprising administering therapeutically effective amounts of:
(a) an FPT inhibitor of the formula: 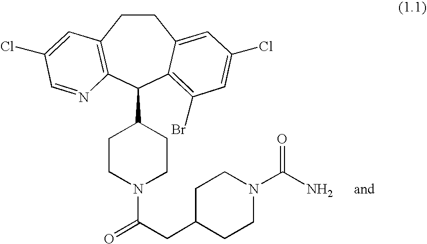
(b) Thalidomide or related imid.

This invention also provides a method of treating multiple myeloma in a patient in need of such treatment comprising administering therapeutically effective amounts of:
(a) an FPT inhibitor of the formula: 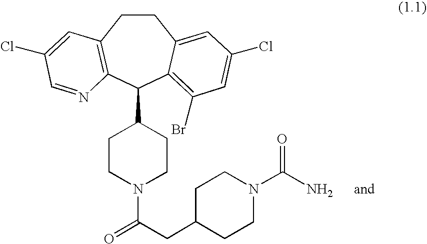
(b) Thalidomide.

This invention is also directed to the methods of treating cancer described herein, particularly those described above, wherein in addition to the administration of the FPT inhibitor and antineoplastic agents radiation therapy is also administered prior to, during, or after the treatment cycle.

The FPT inhibitor and the antineoplastic agents are administered in therapeutically effective dosages to obtain clinically acceptable results, e.g., reduction or elimination of symptoms or of the tumor. Thus, the FPT inhibitor and antineoplastic agents can be administered concurrently or consecutively in a treatment protocol. The administration of the antineoplastic agents can be made according to treatment protocols already known in the art.

The FPT inhibitor and antineoplastic agents are administered in a treatment protocol that usually lasts one to seven weeks, and is repeated typically from 6 to 12 times. Generally the treatment protocol lasts one to four weeks. Treatment protocols of one to three weeks may also be used. A treatment protocol of one to two weeks may also be used. During this treatment protocol or cycle the FPT inhibitor is administered daily while the antineoplastic agents are administered one or more times a week. Generally, the FPT inhibitor can be administered daily (i.e., once per day), preferably twice per day, and the antineoplastic agent is administered once a week or once every three weeks. For example, the taxanes (e.g., Paclitaxel (i.e.,Taxol®),or Docetaxel (i.e., Taxotere®)) can be administered once a week or once every three weeks.

However, those skilled in the art will appreciate that treatment protocols can be varied according to the needs of the patient. Thus, the combination of compounds (drugs) used in the methods of this invention can be administered in variations of the protocols described above. For example, the FPT inhibitor can be administered discontinuously rather than continuously during the treatment cycle. Thus, for example, during the treatment cycle the FPT inhibitor can be administered daily for a week and then discontinued for a week, with this administration repeating during the treatment cycle. Or the FPT inhibitor can be administered daily for two weeks and discontinued for a week, with this administration repeating during the treatment cycle. Thus, the FPT inhibitor can be administered daily for one or more weeks during the cycle and discontinued for one or more weeks during the cycle, with this pattern of administration repeating during the treatment cycle. This discontinuous treatment can also be based upon numbers of days rather than a full week. For example, daily dosing for 1 to 6 days, no dosing for 1 to 6 days with this pattern repeating during the treatment protocol. The number of days (or weeks) wherein the FPT inhibitor is not dosed does not have to equal the number of days (or weeks) wherein the FPT inhibitor is dosed. Usually, if a discontinuous dosing protocol is used, the number of days or weeks that the FPT inhibitor is dosed is at least equal or greater than the number of days or weeks that the FPT inhibitor is not dosed.

The antineoplastic agent could be given by bolus or continuous infusion. The antineoplastic agent could be given daily to once every week, or once every two weeks, or once every three weeks, or once every four weeks during the treatment cycle. If administered daily during a treatment cycle, this daily dosing can be discontinuous over the number of weeks of the treatment cycle. For example, dosed for a week (or a number of days), no dosing for a week (or a number of days, with the pattern repeating during the treatment cycle.

The FPT inhibitor is administered orally, preferably as a solid dosage form, more preferably a capsule, and while the total therapeutically effective daily dose can be administered in one to four, or one to two divided doses per day, generally, the therapeutically effective dose is given once or twice a day, preferably twice a day. The FPT inhibitor can be administered in an amount of about 50 to about 400 mg once per day, and can be administered in an amount of about 50 to about 300 mg once per day. The FPT inhibitor is generally administered in an amount of about 50 to about 350 mg twice a day, usually 50 mg to about 200 mg twice a day, preferably, about 75 mg to about 125 mg administered twice a day, and most preferably about 100 mg administered twice a day.

If the patient is responding, or is stable, after completion of the therapy cycle, the therapy cycle can be repeated according to the judgment of the skilled clinician. Upon completion of the therapy cycles, the patient can be continued on the FPT inhibitor at the same dose that was administered in the treatment protocol, or, if the dose was less than 200 mg twice a day, the dose can be raised to 200 mg twice a day. This maintenance dose can be continued until the patient progresses or can no longer tolerate the dose (in which case the dose can be reduced and the patient can be continued on the reduced dose).

The antineoplastic agents used with the FPT inhibitor are administered in their normally prescribed dosages during the treatment cycle (i.e., the antineoplastic agents are administered according to the standard of practice for the administration of these drugs). For example: (a) about 30 to about 300 mg/m² for the taxanes; (b) about 30 to about 100 mg/m² for Cisplatin; (c) AUC of about 2 to about 8 for Carboplatin; (d) about 2 to about 4 mg/m² for EGF inhibitors that are antibodies; (e) about 50 to about 500 mg/m² for EGF inhibitors that are small molecules; (f) about 1 to about 10 mg/m² for VEGF kinase inhibitors that are antibodies; (g) about 50 to about 2400 mg/m² for VEGF inhibitors that are small molecules; (h) about 1 to about 20 mg for SERMs; (i) about 500 to about 1250 mg/m² for the anti-tumor nucleosides 5-Fluorouracil, Gemcitabine and Capecitabine; (j) for the anti-tumor nucleoside Cytarabine (Ara-C) 100-200 mg/m²/day for 7 to 10 days every 3 to 4 weeks, and high doses for refractory leukemia and lymphoma, i.e., 1 to 3 gm/m² for one hour every 12 hours for 4-8 doses every 3 to four weeks; (k) for the anti-tumor nucleoside Fludarabine (F-ara-A) 10-25 mg/m²/day every 3 to 4 weeks; (l) for the anti-tumor nucleoside Decitabine 30 to 75 mg/m² for three days every 6 weeks for a maximum of 8 cycles; (m) for the anti-tumor nucleoside Chlorodeoxyadenosine (CdA, 2-CdA) 0.05-0.1 mg/kg/day as continuous infusion for up to 7 days every 3 to 4 weeks; (n) about 1 to about 1.00 mg/m² for epothilones; (o) about 1 to about 350 mg/m2 for topoisomerase inhibitors; (p) about 1 to about 50 mg/m2 for vinca alkaloids; (q) for the folate antagonist Methotrexate (MTX) 20-60 mg/m² by oral, IV or IM every 3 to 4 weeks, the intermediate dose regimen is 80-250 mg/m² IV over 60 minutes every 3 to 4 weeks, and the high dose regimen is 250-1000mg/m² IV given with leucovorin every 3 to 4 weeks; (r) for the folate antagonist Premetrexed (Alimta) 300-600 mg/m² (10 minutes IV infusion day 1) every 3 weeks; (s) for the ribonucleotide reductase inhibitor Hydroxyurea (HU) 20-50 mg/kg/day (as needed to bring blood cell counts down); (t) the platinum coordinator compound Oxaliplatin (Eloxatin) 50-100 mg/m² every 3 to 4 weeks (preferably used for solid tumors such as non-small cell lung cancer, colorectal cancer and ovarian cancer); (u) for the anthracycline daunorubicin 10-50 mg/m²/day IV for 3-5 days every 3 to 4 weeks; (v) for the anthracycline Doxorubicin (Adriamycin) 50-100 mg/m² IV continuous infusion over 1-4 days every 3 to 4 weeks, or 10-40 mg/m² IV weekly; (w) for the anthracycline Idarubicin 10-30 mg/m² daily for 1-3 days as a slow IV infusion over 10-20 minutes every 3 to 4 weeks; (x) for the biologic interferon (Intron-A, Roferon) 5 to 20 million IU three times per week; (y) for the biologic pegylated interferon (Peg-intron, Pegasys) 3 to 4 micrograms/kg/day chronic sub cutaneous (until relapse or loss of activity); and (z) for the biologic Rituximab (Rituxan) (antibody used for non-Hodgkin's lymphoma) 200-400mg/m²IV weekly over 4-8 weeks for 6 months.

Gleevec can be used orally in an amount of about 200 to about 800 mg/day.

Thalidomide (and related imids) can be used orally in amounts of about 200 to about 800 mg/day, and can be contiuously dosed or used until releapse or toxicity. See for example Mitsiades et al., “Apoptotic signaling induced by immunomodulatory thalidomide analoqs in human multiple myeloma cells; therapeutic implications”, Blood, 99(12):4525-30, Jun. 15, 2002, the disclosure of which is incorporated herein by reference thereto

For example, Paclitaxel (e.g., Taxol®) can be administered once per week in an amount of about 50 to about 100 mg/m² with about 60 to about 80 mg/m² being preferred. In another example Paclitaxel (e.g., Taxol®) can be administered once every three weeks in an amount of about 150 to about 250 mg/m with about 175 to about 225 mg/m² being preferred.

In another example, Docetaxel (e.g., Taxotere®) can be administered once per week in an amount of about 10 to about 45 mg/m². In another example Docetaxel (e.g., Taxotere®) can be administered once every three weeks in an amount of about 50 to about 100 mg/m².

In another example Cisplatin can be administered once per week in an amount of about 20 to about 40 mg/m². In another example Cisplatin can be administered once every three weeks in an amount of about 60 to about 100 mg/m².

In another example Carboplatin can be administered once per week in an amount to provide an AUC of about 2 to about 3. In another example Carboplatin can be administered once every three weeks in an amount to provide an AUC of about 5 to about 8.

Thus, in one example (e.g., treating non small cell lung cancer):
(1) the FPT inhibitor is administered in an amount of about 50 mg to about 200 mg twice a day, preferably, about 75 mg to about 125 mg administered twice a day, and most preferably about 100 mg administered twice a day;
(2) Paclitaxel (e.g., Taxol®) is administered once per week in an amount of about 50 to about 100 mg/m² with about 60 to about 80 mg/m² being preferred; and
(3) Carboplatin is administered once per week in an amount to provide an AUC of about 2 to about 3.

In another example (e.g., treating non small cell lung cancer):
(1) the FPT inhibitor is administered in an amount of about 50 mg to about 200 mg twice a day, preferably, about 75 mg to about 125 mg administered twice a day, and most preferably about 100 mg administered twice a day;
(2) Paclitaxel (e.g., Taxol®) is administered once per week in an amount of about 50 to about 100 mg/m² with about 60 to about 80 mg/m² being preferred; and
(3) Cisplatin is administered once per week in an amount of about 20 to about 40 Mg/m^{2.}

In another example (e.g., treating non small cell lung cancer):
(1) the FPT inhibitor is administered in an amount of about 50 mg to about 200 mg twice a day, preferably, about 75 mg to about 125 mg administered twice a day, and most preferably about 100 mg administered twice a day;
(2) Docetaxel (e.g., Taxotere®) is administered once per week in an amount of about 10 to about 45 mg/m²; and
(3) Carboplatin is administered once per week in an amount to provide an AUC of about 2 to about 3.

In another example (e.g., treating non small cell lung cancer):
(1) the FPT inhibitor is administered in an amount of about 50 mg to about 200 mg twice a day, preferably, about 75 mg to about 125 mg administered twice a day, and most preferably about 100 mg administered twice a day;
(2) Docetaxel (e.g., Taxotere®) is administered once per week in an amount of about 10 to about 45 mg/m²; and
(3) Cisplatin is administered once per week in an amount of about 20 to about 40 mg/m^{2.}

Thus, in one example (e.g., treating non small cell lung cancer):
(1) the FPT inhibitor is administered in an amount of about 50 mg to about 200 mg twice a day, preferably, about 75 mg to about 125 mg administered twice a day, and most preferably about 100 mg administered twice a day;
(2) Paclitaxel (e.g., Taxol®) is administered once every three weeks in an amount of about 150 to about 250 mg/m², with about 175 to about 225 mg/m² being preferred, and with 175 mg/m² being most preferred; and
(3) Carboplatin is administered once every three weeks in an amount to provide an AUC of about 5 to about 8, and preferably 6.

In a preferred example of treating non small cell lung cancer:
(1) the FPT inhibitor is administered in an amount of 100 mg administered twice a day;
(2) Paclitaxel (e.g., Taxol®) is administered once every three weeks in an amount of 175 mg/m²; and
(3) Carboplatin is administered once every three weeks in an amount to provide an AUC of 6.

In another example (e.g., treating non small cell lung cancer):
(1) the FPT inhibitor is administered in an amount of about 50 mg to about 200 mg twice a day, preferably, about 75 mg to about 125 mg administered twice a day, and most preferably about 100 mg administered twice a day;
(2) Paclitaxel (e.g., Taxol®) is administered once every three weeks in an amount of about 150 to about 250 mg/m², with about 175 to about 225 mg/m² being preferred; and
(3) Cisplatin is administered once every three weeks in an amount of about 60 to about 100 mg/m².

In another example (e.g., treating non small cell lung cancer):
(1) the FPT inhibitor is administered in an amount of about 50 mg to about 200 mg twice a day, preferably, about 75 mg to about 125 mg administered twice a day, and most preferably about 100 mg administered twice a day;
(2) Docetaxel (e.g., Taxotere®) is administered once every three weeks in an amount of about 50 to about 100 mg/m²; and
(3) Carboplatin is administered once every three weeks in an amount to provide an AUC of about 5 to about 8.

In another example (e.g., treating non small cell lung cancer):
(1) the FPT inhibitor is administered in an amount of about 50 mg to about 200 mg twice a day, preferably, about 75 mg to about 125 mg administered twice a day, and most preferably about 100 mg administered twice a day;
(2) Docetaxel (e.g., Taxotere®) is administered once every three weeks in an amount of about 50 to about 100 mg/m²; and
(3) Cisplatin is administered once every three weeks in an amount of about 60 to about 100 mg/m².

In a preferred example for treating non small cell lung cancer using the FPT inhibitor, Docetaxel and Carboplatin:
(1) the FPT inhibitor is administered in an amount of about 50 mg to about 200 mg twice a day, preferably, about 75 mg to about 125 mg administered twice a day, and most preferably about 100 mg administered twice a day;
(2) Docetaxel (e.g., Taxotere®) is administered once every three weeks in an amount of about 75 mg/m²; and
(3) Carboplatin is administered once every three weeks in an amount to provide an AUC of about 6.

In the above examples the Docetaxel (e.g., Taxotere®) and Cisplatin, the Docetaxel (e.g., Taxotere®) and Carboplatin, the Paclitaxel (e.g., Taxol®) and Carboplatin, or the Paclitaxel (e.g., Taxol®) and Cisplatin are preferably administered on the same day.

In another example (e.g., CML):
(1) the FPT inhibitor is administered in an amount of about 100 mg to about 200 mg administered twice a day;
(2) Gleevec is administered in an amount of about 400 to about 800 mg/day orally; and
(3) interferon (Intron-A) is administered in an amount of about 5 to about 20 million IU three times per week.

In another example (e.g., CML):
(1) the FPT inhibitor is administered in an amount of about 100 mg to about 200 mg administered twice a day;
(2) Gleevec is administered in an amount of about 400 to about 800 mg/day orally; and
(3) pegylated interferon (Peg-Intron or Pegasys) is administered in an amount of about 3 to about 6 micrograms/kg/day.

In another example (e.g., non-Hodgkin's lymphoma):
(1) the FPT inhibitor is administered in an amount of about 50 mg to about 200 mg twice a day, preferably, about 75 mg to about 125 mg administered twice a day, and most preferably about 100 mg administered twice a day; and
(2) Genasense (antisense to BCL-2) is administered as a continuous IV infusion at a dose of about 2 to about 5 mg/kg/day (e.g., 3 mg/kg/day) for 5 to 7 days every 3 to 4 weeks.

In another example (e.g., multiple myeloma):
(1) the FPT inhibitor is administered in an amount of about 50 mg to about 200 mg twice a day, preferably, about 75 mg to about 125 mg administered twice a day, and most preferably about 100 mg administered twice a day; and
(2) the proteosome inhibitor (e.g., PS-341—Millenium) is administered in an amount of about 1.5mg/m² twice weekly for two consecutive weeks with a one week rest period.

In another example (e.g., multiple myeloma):
(1) the FPT inhibitor is administered in an amount of about 50 mg to about 200 mg twice a day, preferably, about 75 mg to about 125 mg administered twice a day, and most preferably about 100 mg administered twice a day; and
(2) the Thalidomide (or related imid) is administered orally in an amount of about 200 to about 800 mg/day, with dosing being continuous until relapse or toxicity.

If the patient is responding, or is stable, after completion of the therapy cycle, the therapy cycle can be repeated according the judgment of the skilled clinician. Upon completion of the therapy cycles, the patient can be continued on the FPT inhibitor at the same dose that was administered in the treatment protocol, or, if the dose was less than 200mg twice a day, the dose can be raised to 200 mg twice a day. This maintenance dose can be continued until the patient progresses or can no longer tolerate the dose (in which case the dose can be reduced and the patient can be continued on the reduced dose).

The cancers which can be treated in the methods of this invention include, but are not limited to: lung cancers (e.g., non small cell lung cancer), head and/or neck cancers (e.g. squamous cell cancer of the head or neck), ovarian cancers, breast cancers, bladder cancers, and prostate cancers.

Cancers which may be treated by the methods of this invention are: colorectal cancers, pancreatic cancers, thyroid follicular cancers, anaplastic thyroid carcinoma, non-Hodgkin's lymphoma, myelodysplastic syndrome (MDS), CMML (chronic myelomonocytic leukemia), AML, ALL (acute lymphoid leukemia, e.g., ALL PH+), CML, myeloma (e.g., multiple myeloma), cancers of mesenchymal origin (e.g., fibrosarcomas and rhabdomyosarcomas), melanomas, teratocarcinomas, neuroblastomas, gliomas, kidney carcinomas and hepatomas.

Antineoplastic agents that can be used in combination with the FPT inhibitor are:
(1) taxanes such as Paclitaxel (Taxol®) and/or Docetaxel (e.g., Taxotere®);
(2) platinum coordinator compounds, such as, for example, Carboplatin, Cisplatin and Oxaliplatin (e.g., Eloxatin);
(3) EGF inhibitors that are antibodies, such as: HER2 antibodies (such as, for example trastuzumab (Herceptin®), Genentech, Inc.), Cetuximab (Erbitux, IMC-C225, ImClone Systems), EMD 72000 (Merck KGaA), anti-EFGR monoclonal antibody ABX (Abgenix), TheraCIM-h-R3 (Center of Molecular Immunology), monoclonal antibody 425 (Merck KGaA), monoclonal antibody ICR-62 (ICR, Sutton, England); Herzyme (Elan Pharmaceutical Technologies and Ribozyme Pharmaceuticals), PKI 166 (Novartis), EKB 569 (Wyeth-Ayerst), GW 572016 (GlaxoSmithKline), Cl 1033 (Pfizer Global Research and Development), Trastuzmab-maytansinoid conjugate (Genentech, Inc.), Mitumomab (Imclone Systems and Merck KGaA) and Melvax II (Imclone Systems and Merck KgaA);
(4) EGF inhibitors that are small molecules, such as, Tarceva (TM) (OSI-774, OSI Pharmaceuticals, Inc.), and Iressa (ZD 1839, Astra Zeneca);
(5) VEGF inhibitors that are antibodies such as: Bevacizumab (Genentech, Inc.), and IMC-1C11 (ImClone Systems), DC 101 (a KDR VEGF Receptor 2 from ImClone Systems);
(6) VEGF kinase inhibitors that are small molecules such as SU 5416 and SU 6688 (both from Sugen, Inc.);
(7) estrogen receptor antagonists or selective estrogen receptor modulators (SERMs), such as Tamoxifen, Idoxifene, Raloxifene, trans-2,3-Dihydroraloxifene, Levormeloxifene, Droloxifene, MDL 103,323, and Acolbifene (Schering Corp.);
(8) anti-tumor nucleoside derivatives such as 5-Fluorouracil, Gemcitabine, Capecitabine, Cytarabine (Ara-C), Fludarabine (F-Ara-A), Decitabine, and Chlorodeoxyadenosine (CdA, 2-CdA);
(9) epothilones such as BMS-247550 (Bristol-Myers Squibb), and EPO906 (Novartis Pharmaceuticals);
(10) topoisomerase inhibitors such as Topotecan (Glaxo SmithKline), and Camptosar (Pharmacia);
(11) vinca alkaloids, such as, Navelbine (Anvar and Fabre, France), Vincristine and Vinblastine;
(12) antibodies that are inhibitors of αVβ3 integrins, such as, LM-609 (see, Clinical Cancer Research, Vol. 6, page 3056-3061, August 2000, the disclosure of which is incorporated herein by reference thereto);
(13) folate antagonists, such as Methotrexate (MTX), and Premetrexed (Alimta);
(14) ribonucleotide reductase inhibitors, such as Hydroxyurea (HU);
(15) anthracyclines, such as Daunorubicin, Doxorubicin (Adriamycin), and Idarubicin; and
(16) biologics, such as interferon (e.g., Intron-A and Roferon), pegylated interferon (e.g., Peg-Intron and Pegasys), and Rituximab (Rituxan, antibody used for the treatment of non-Hodgkin's lymphoma).

Preferred antineoplastic agents are selected from: Paclitaxel, Docetaxel, Carboplatin, Cisplatin, Gemcitabine, Tamoxifen, Herceptin, Cetuximab, Tarceva, Iressa, bevacizumab, Navelbine, IMC-1C11, SU5416 or SU6688. Most preferred antineoplastic agents are selected from: Paclitaxel, Docetaxel, Carboplatin, Cisplatin, Navelbine, Gemcitabine, or Herceptin.

In general when more than one antineoplastic agent is used in the methods of this invention, the antineoplastic agents are administered on the same day either concurrently or consecutively in their standard dosage form. For example, the antineoplastic agents are usually administered intravenously, preferably by an IV drip using IV solutions well known in the art (e.g., isotonic saline (0.9% NaCl) or dextrose solution (e.g., 5% dextrose)).

When two or more antineoplastic agents are used, the antineoplastic agents are generally administered on the same day; however, those skilled in the art will appreciate that the antineoplastic agents can be administered on different days and in different weeks. The skilled clinician can administer the antineoplastic agents according to their recommended dosage schedule from the manufacturer of the agent and can adjust the schedule according to the needs of the patient, e.g., based on the patient's response to the treatment. For example, when Gemcitabine is used in combination with a platinum coordinator compound, such as, for example, Cisplatin, to treat lung cancer, both the Gemcitabine and the Cisplatin are given on the same day on day one of the treatment cycle, and then Gemcitabine is given alone on day 8 and given alone again on day 15

Thus, one embodiment of this invention is directed to a method of treating cancer comprising administering to a patient in need of such treatment therapeutically effective amounts of the FPT inhibitor (1.0 or 1.1), a taxane, and a platinum coordination compound.

Another embodiment of this invention is directed to a method of treating cancer comprising administering to a patient in need of such treatment therapeutically effective amounts of the FPT inhibitor (1.0 or 1.1), a taxane, and a platinum coordination compound, wherein said FPT inhibitor is administered every day, said taxane is administered once per week per cycle, and said platinum coordinator compound is administered once per week per cycle. Preferably the treatment is for one to four weeks per cycle.

Another embodiment of this invention is directed to a method of treating cancer comprising administering to a patient in need of such treatment therapeutically effective amounts of the FPT inhibitor (1.0 or 1.1), a taxane, and a platinum coordination compound, wherein said FPT inhibitor is administered every day, said taxane is administered once every three weeks per cycle, and said platinum coordinator compound is administered once every three weeks per cycle. Preferably the treatment is for one to three weeks per cycle.

Another embodiment of this invention is directed to a method of treating cancer comprising administering to a patient in need of such treatment therapeutically effective amounts of the FPT inhibitor (1.0 or 1.1), Paclitaxel, and Carboplatin. Preferably, said FPT inhibitor is administered every day, said Paclitaxel is administered once per week per cycle, and said Carboplatin is administered once per week per cycle. Preferably the treatment is for one to four weeks per cycle.

Another embodiment of this invention is directed to a method of treating cancer comprising administering to a patient in need of such treatment therapeutically effective amounts of the FPT inhibitor (1.0 or 1.1), Paclitaxel, and Carboplatin. Preferably, said FPT inhibitor is administered every day, said Paclitaxel is administered once every three weeks per cycle, and said Carboplatin is administered once every three weeks per cycle. Preferably the treatment is for one to three weeks per cycle.

Preferably, non small cell lung cancer is treated in the methods described in the above embodiments.

Another embodiment of this invention is directed to a method for treating non small cell lung cancer in a patient in need of such treatment comprising administering daily a therapeutically effective amount of the FPT inhibitor (1.0 or 1.1), administering a therapeutically effective amount of Carboplatin once a week per cycle, and administering a therapeutically effective amount of Paclitaxel once a week per cycle, wherein the treatment is given for one to four weeks per cycle. Preferably said FPT inhibitor is administered twice per day. Preferably said Carboplatin and said Paclitaxel are administered on the same day, and more preferably said Carboplatin and said Paclitaxel are administered consecutively, and most preferably said Carboplatin is administered after said Paclitaxel.

Another embodiment of this invention is directed to a method for treating non small cell lung cancer in a patient in need of such treatment comprising administering daily a therapeutically effective amount of the FPT inhibitor (1.0 or 1.1), administering a therapeutically effective amount of Carboplatin once every three weeks per cycle, and administering a therapeutically effective amount of Paclitaxel once every three weeks per cycle, wherein the treatment is given for one to three weeks. Preferably said FPT inhibitor is administered twice per day. Preferably said Carboplatin and said Paclitaxel are administered on the same day, and more preferably said Carboplatin and said Paclitaxel are administered consecutively, and most preferably said Carboplatin is administered after said Paclitaxel.

A preferred embodiment of this invention is directed to a method for treating non small cell lung cancer in a patient in need of such treatment comprising administering about 50 to about 200 mg of the FPT inhibitor (1.0 or 1.1) twice a day, administering Carboplatin once per week per cycle in an amount to provide an AUC of about 2 to about 8 (preferably about 2 to about 3), and administering once per week per cycle about 60 to about 300 mg/m² (preferably about 50 to 100 mg/m², more preferably about 60 to about 80 mg/m²) of Paclitaxel, wherein the treatment is given for one to four weeks per cycle. In a more preferred embodiment said FPT inhibitor is administered in amount of about 75 to about 125 mg twice a day, with about 100 mg twice a day being preferred. Preferably said Carboplatin and said Paclitaxel are administered on the same day, and more preferably said Carboplatin and said Paclitaxel are administered consecutively, and most preferably said Carboplatin is administered after said Paclitaxel.

In another preferred embodiment, this invention is directed to a method for treating non small cell lung cancer in a patient in need of such treatment comprising administering about 50 to about 200 mg of the FPT inhibitor (1.0 or 1.1) twice a day, administering Carboplatin once every three weeks per cycle in an amount to provide an AUC of about 2 to about 8 (preferably about 5 to about 8), and administering once every three weeks per cycle about 150 to about 225 mg/m² (preferably about 175 to about 225 mg/m²) of Paclitaxel, wherein the treatment is given for one to three weeks. In a more preferred embodiment said FPT inhibitor is administered in an amount of about 75 to about 125 mg twice a day, with about 100 mg twice a day being preferred. Preferably said Carboplatin and said Paclitaxel are administered on the same day, and more preferably said Carboplatin and said Paclitaxel are administered consecutively, and most preferably said Carboplatin is administered after said Paclitaxel.

In a still more preferred embodiment, this invention is directed to a method for treating non small cell lung cancer in a patient in need of such treatment comprising administering 100 mg of the FPT inhibitor (1.0 or 1.1) twice a day, administering Carboplatin once every three weeks per cycle in an amount to provide an AUC of 6, and administering once every three weeks per cycle 175 mg/m² of Paclitaxel, wherein the treatment is given for one to three weeks. Preferably said Carboplatin and said Paclitaxel are administered on the same day, and more preferably said Carboplatin and said Paclitaxel are administered consecutively, and most preferably said Carboplatin is administered after said Paclitaxel.

Other embodiments of this invention are directed to methods of treating cancer as described in the above embodiments except that in place of Paclitaxel and Carboplatin the taxanes and platinum coordinator compounds used together in the methods are: (1) Docetaxel (Taxotere®) and Cisplatin; (2) Paclitaxel and Cisplatin; and (3) Docetaxel and Carboplatin. In the methods of this invention Cisplatin is preferably used in amounts of about 30 to about 100 mg/m². In the methods of this invention Docetaxel is preferably used in amounts of about 30 to about 100 mg/m².

In another embodiment this invention is directed to a method of treating cancer comprising ad ministering to a patient in need of such treatment therapeutically effective amounts of the FPT inhibitor (1.0 or 1.1), a taxane, and an EGF inhibitor that is an antibody. Preferably the taxane used is Paclitaxel, and preferably the EGF inhibitor is a HER2 antibody (more preferably Herceptin) or Cetuximab, and most preferably Herceptin is used. The length of treatment, and the amounts and administration of the FPT inhibitor and the taxane are as described in the embodiments above. The EGF inhibitor that is an antibody is administered once a week per cycle, and is preferably administered on the same day as the taxane, and more preferably is administered consecutively with the taxane. For example, Herceptin is administered in a loading dose of about 3 to about 5 mg/m² (preferably about 4 mg/m²), and then is administered in a maintenance dose of about 2 mg/m² once per week per cycle for the remainder of the treatment cycle (usually the cycle is 1 to 4 weeks). Preferably the cancer treated is breast cancer.

In another embodiment this invention is directed to a method of treating cancer comprising administering to a patient in need of such treatment therapeutically effective amounts of:

(1) the FPT inhibitor (1.0 or 1.1);

(2) a taxane; and

(3) an antineoplastic agent selected from:
(a) an EGF inhibitor that is a small molecule;
(b) a VEGF inhibitor that is an antibody; or
(c) a VEGF kinase inhibitor that is a small molecule.
   Preferably, the taxane Paclitaxel or Docetaxel is used. Preferably the antineoplastic agent is selected from: Tarceva, Iressa, Bevacizumab, SU5416 or SU6688. The length of treatment, and the amounts and administration of the FPT inhibitor and the taxane are as described in the embodiments above. The VEGF kinase inhibitor that is an antibody is usually given once per week per cycle. The EGF and VEGF inhibitors that are small molecules are usually given daily per cycle. Preferably, the VEGF inhibitor that is an antibody is given on the same day as the taxane, and more preferably is administered concurrently with the taxane. When the EGF inhibitor that is a small molecule or the VEGF inhibitor that is a small molecule is administered on the same day as the taxane, the administration is preferably concurrently with the taxane. The EGF or VEGF kinase inhibitor is generally administered in an amount of about 10 to about 500 mg/m². Preferably the cancer treated is non small cell lung cancer.

In another embodiment this invention is directed to a method of treating cancer comprising administering to a patient in need of such treatment therapeutically effective amounts of the FPT inhibitor (1.0 or 1.1), an anti-tumor nucleoside derivative, and a platinum coordination compound.

Another embodiment of this invention is directed to a method of treating cancer comprising administering to a patient in need of such treatment therapeutically effective amounts of the FPT inhibitor (1.0 or 1.1), an anti-tumor nucleoside derivative, and a platinum coordination compound, wherein said FPT inhibitor is administered every day, said anti-tumor nucleoside derivative is administered once per week per cycle, and said platinum coordinator compound is administered once per week per cycle. Although the treatment can be for one to four weeks per cycle, the treatment is preferably for one to seven weeks per cycle.

Another embodiment of this invention is directed to a method of treating cancer comprising administering to a patient in need of such treatment therapeutically effective amounts of the FPT inhibitor (1.0 or 1.1), an anti-tumor nucleoside. derivative, and a platinum coordination compound, wherein said FPT inhibitor is administered every day, said an anti-tumor nucleoside derivative is administered once per week per cycle, and said platinum coordinator compound is administered once every three weeks per cycle. Although the treatment can be for one to four weeks per cycle, the treatment is preferably for one to seven weeks per cycle.

Another embodiment of this invention is directed to a method of treating cancer comprising administering to a patient in need of such treatment therapeutically effective amounts of the FPT inhibitor (1.0 or 1.1), Gemcitabine, and Cisplatin. Preferably, said FPT inhibitor is administered every day, said Gemcitabine is administered once per week per cycle, and said Cisplatin is administered once per week per cycle. Preferably the treatment is for one to seven weeks per cycle.

Another embodiment of this invention is directed to a method of treating cancer comprising administering to a patient in need of such treatment therapeutically effective amounts of the FPT inhibitor (1.0 or 1.1), Gemcitabine, and Cisplatin. Preferably, said FPT inhibitor is administered every day, said Gemcitabine is administered once per week per cycle, and said Cisplatin is administered once every three weeks per cycle. Preferably the treatment is for one to seven weeks.

Another embodiment of this invention is directed to a method of treating cancer comprising administering to a patient in need of such treatment therapeutically effective amounts of the FPT inhibitor (1.0 or 1.1), Gemcitabine, and Carboplatin.

Preferably, said FPT inhibitor is administered every day, said Gemcitabine is administered once per week per cycle, and said Carboplatin is administered once per week per cycle. Preferably the treatment is for one to seven weeks per cycle.

Another embodiment of this invention is directed to a method of treating cancer comprising administering to a patient in need of such treatment therapeutically effective amounts of the FPT inhibitor (1.0 or 1.1), Gemcitabine, and Carboplatin. Preferably, said FPT inhibitor is administered every day, said Gemcitabine is administered once per week per cycle, and said Carboplatin is administered once every three weeks per cycle. Preferably the treatment is for one to seven weeks per cycle.

Preferably, non small cell lung cancer is treated in the methods using gemcitabine in the embodiments described above.

In the above embodiments using Gemcitabine, the FPT inhibitor and the platinum coordinator compound are administered as described above for the embodiments using taxanes. Gemcitabine is administered in an amount of about 500 to about 1250 mg/m². The Gemcitabine is preferably administered on the same day as the platinum coordinator compound, and more preferably consecutively with the platinum coordinator compound, and most preferably the Gemcitabine is administered after the platinum coordinator compound.

Another embodiment of this invention is directed to a method of treating cancer in a patient in need of such treatment comprising administering the FPT inhibitor (1.0 or 1.1) and an antineoplastic agent selected from: (1) EGF inhibitors that are antibodies, (2) EGF inhibitors that are small molecules, (3) VEGF inhibitors that are antibodies, and (4) VEGF kinase inhibitors that are small molecules all as described above. The treatment is for one to seven weeks per cycle, and generally for one to four weeks per cycle. The FPT inhibitor is administered in the same manner as described above for the other embodiments of this invention. The small molecule antineoplastic agents are usually administered daily, and the antibody antineoplastic agents are usually administered once per week per cycle. The antineoplastic agents are preferably selected from: Herceptin, Cetuximab, Tarceva, Iressa, bevacizumab, IMC-1C11, SU5416 or SU6688. Preferably non small cell lung cancer is treated.

In the embodiments of this invention wherein a platinum coordinator compound is used as well as at least one other antineoplastic agent, and these drugs are administered consecutively, the platinum coordinator compound is generally administered after the other antineoplastic agents have been administered.

Other embodiments of this invention include the administration of a therapeutically effective amount of radiation to the patient in addition to the administration of the FPT inhibitor and antineoplastic agents in the embodiments described above. Radiation is administered according to techniques and protocols well know to those skilled in the art.

Another embodiment of this invention is directed to a pharmaceutical composition comprising at least two different antineoplastic agents and a pharmaceutically acceptable carrier for intravenous administration. Preferably the pharmaceutically acceptable carrier is an isotonic saline solution (0.9% NaCl) or a dextrose solution (e.g., 5% dextrose).

Another embodiment of this invention is directed to a pharmaceutical composition comprising the FPT inhibitor and at least two different antineoplastic agents and a pharmaceutically acceptable carrier for intravenous. administration. Preferably the pharmaceutically acceptable carrier is an isotonic saline solution (0.9% NaCl) or a dextrose solution (e.g., 5% dextrose).

Another embodiment of this invention is directed to a pharmaceutical composition comprising the FPT inhibitor and at least one antineoplastic agent and a pharmaceutically acceptable carrier for intravenous administration. Preferably the pharmaceutically acceptable carrier is an isotonic saline solution (0.9% NaCl) or a dextrose solution (e.g., 5% dextrose).

Those skilled in the art will appreciate that the compounds (drugs) used in the methods of this invention are available to the skilled clinician in pharmaceutical compositions (dosage forms) from the manufacture and are used in those compositions. So, the recitation of the compound or class of compounds in the above described methods can be replaced with a recitation of a pharmaceutical composition comprising the particular compound or class of compounds. For example, the embodiment directed to a method of treating cancer comprising administering to a patient in need of such treatment therapeutically effective amounts of the FPT inhibitor (1.0 or 1.1), a taxane, and a platinum coordination compound, includes within its scope a method of treating cancer comprising administering to a patient in need of such treatment therapeutically effective amounts of a pharmaceutical composition comprising the FPT inhibitor (1.0 or 1.1), a pharmaceutical composition comprising a taxane, and a pharmaceutical composition comprising a platinum coordination compound.

The actual dosage employed may be varied depending upon the requirements of the patient and the severity of the condition being treated. Determination of the proper dosage for a particular situation is within the skill of the art.

The amount and frequency of administration of the FPT inhibitor and the antineoplastic agents will be regulated according to the judgment of the attending clinician (physician) considering such factors as age, condition and size of the patient as well as severity of the cancer being treated.

The antineoplastic agent can be administered according to therapeutic protocols well known in the art. It will be apparent to those skilled in the art that the administration of the antineoplastic agent can be varied depending on the cancer being treated and the known effects of the antineoplastic agent on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the cancer to the administered therapeutic agents.

The initial administration can be made according to established protocols known in the art, and then, based upon the observed effects, the dosage, modes of administration and times of administration can be modified by the skilled clinician.

The particular choice of antineoplastic agent will depend upon the diagnosis of the attending physicians and their judgement of the condition of the patient and the appropriate treatment protocol.

The determination of the order of administration, and the number of repetitions of administration of the antineoplastic agent during a treatment protocol, is well within the knowledge of the skilled physician after evaluation of the cancer being treated and the condition of the patient.

Thus, in accordance with experience and knowledge, the practicing physician can modify each protocol for the administration of an antineoplastic agent according to the individual patient's needs, as the treatment proceeds. All such modifications are within the scope of the present invention.

The attending clinician, in judging whether treatment is effective at the dosage administered, will consider the general well-being of the patient as well as more definite signs such as relief of cancer-related symptoms (e.g., pain, cough (for lung cancer), and shortness of breath (for lung cancer)), inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A method of treating cancer comprising administering to a patient in need of such treatment therapeutically effective amounts of:
(a) the FPT inhibitor 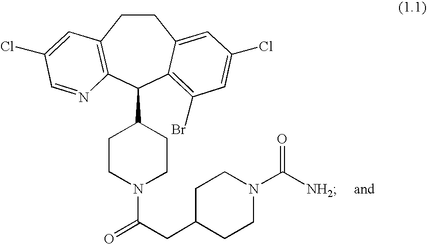
(b) at least two different antineoplastic agents selected from the group consisting of:
(1) taxanes;
(2) platinum coordinator compounds;
(3) EGF inhibitors that are antibodies;
(4) EGF inhibitors that are small molecules;
(5) VEGF inhibitors that are antibodies;
(6) VEGF kinase inhibitors that are small molecules;
(7) estrogen receptor antagonists or selective estrogen receptor modulators;
(8) anti-tumor nucleoside derivatives;
(9) epothilones;
(10) topoisomerase inhibitors;
(11) vinca alkaloids;
(12) antibodies that are inhibitors of αVβ3 integrins;
(13) small molecule inhibitors of αVβ3 integrins;
(14) folate antagonists;
(15) ribonucleotide reductase inhibitors;
(16) anthracyclines;
(17) biologics;
(18) Thalidomide (or related Imid); and
(19) Gleevec.

2. The method of claim 1 wherein two antineoplastic agents are used wherein one antineoplastic agent is a taxane, and the other antineoplastic agent is a platinum coordinator compound.

3. The method of claim 2 wherein said taxane is selected from Paclitaxel or Docetaxel, and said platinum coordinator compound is selected from Carboplatin or Cisplatin.

4. The method of claim 2 wherein said taxane is Paclitaxel and said platinum coordinator compound is Carboplatin.

5. The method of claim 2 wherein said taxane is Paclitaxel and said platinum coordinator compound is Cisplatin.

6. The method of claim 2 wherein said taxane is Docetaxel and said platinum coordinator compound is Cisplatin.

7. The method of claim 2 wherein said taxane is Docetaxel and said platinum coordinator compound is Carboplatin.

8. The method of claim 2 wherein: said taxane is Paclitaxel administered in an amount of about 150 mg to about 300 mg/m² once every three weeks per cycle, and said platinum coordinator compound is Carboplatin administered once every three weeks per cycle in amount of to provide an AUC of about 5 to about 8.

9. The method of claim 2 wherein: said taxane is Docetaxel administered in an amount of about 50 mg to about 100 mg/m² once every three weeks per cycle, and said platinum coordinator compound is Cisplatin administered in amount of about 60 mg to about 100 mg/m² once every three weeks per cycle.

10. The method of claim 2 wherein said FPT inhibitor is administered in an amount of about 50 mg to about 200 mg twice a day.

11. The method of claim 10 wherein said FPT inhibitor is administered in an amount of about 75 mg to about 125 mg twice a day.

12. The method of claim 2 wherein the treatment is given for one to four weeks per cycle.

13. The method of claim 2 wherein non small cell lung cancer is treated.

14. The method of claim 1 wherein two antineoplastic agents are used wherein one antineoplastic agent is a taxane, and the other antineoplastic agent is an EGF inhibitor that is an antibody.

15. The method of claim 14 wherein said taxane is Paclitaxel and said EGF inhibitor is Herceptin.

16. The method of claim 1 wherein two antineoplastic agents are used and wherein one antineoplastic agent is an antinucleoside derivative, and the other antineoplastic agent is a platinum coordinator compound.

17. The method of claim 16 wherein said antinucleoside derivative is Gemcitabine and said platinum coordinator compound is Cisplatin.

18. The method of claim 16 wherein said antinucleoside derivative is Gemcitabine and said platinum coordinator compound is Carboplatin.

19. A method of treating non small cell lung cancer in a patient in need of such treatment comprising administering therapeutically effective amounts of:
(a) an FPT inhibitor of the formula: 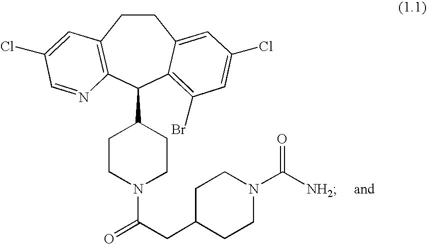
(b) Carboplatin; and
(c) Paclitaxel.

20. The method of claim 19 wherein said FPT inhibitor is administered twice a day, said carboplatin is administered once every three weeks per cycle, and said paclitaxel is administered once every three weeks per cycle, said treatment being given for one to four weeks per cycle.

21. The method of claim 20 wherein said FPT inhibitor is administered in an amount of about 50 mg to about 200 mg twice a day, said carboplatin is administered once every three weeks per cycle in an amount to provide an AUC of about 5 to about 8, said paclitaxel is administered once every three weeks per cycle in an amount of about 150 to about 300 mg/m², and wherein said carboplatin and said paclitaxel are administered on the same day.

22. The method of claim 21 wherein said FPT inhibitor is administered in an amount of about 75 mg to about 125 mg twice a day.

23. The method of claim 22 wherein said FPT inhibitor is administered in an amount of about 100 mg twice a day.

24. The method of claim 19 wherein:
(1) said FPT inhibitor is administered in an amount of about 100 mg twice a day;
(2) said Carboplatin is administered once every three weeks per cycle in an amount to provide an AUC of about 6; and
(3) said Paclitaxel is administered once every three weeks per cycle in an amount of about 175 mg/m².

25. The method of claim 24 wherein said Carboplatin and said Paclitaxel are administered on the same day

26. A method of treating non small cell lung cancer in a patient in need of such treatment comprising administering therapeutically effective amounts of:
(a) an FPT inhibitor of the formula: 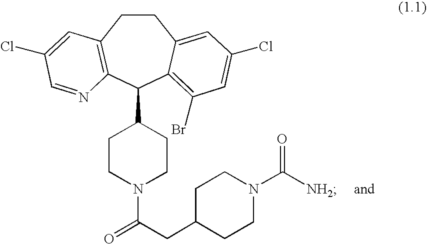
(b) Cisplatin; and
(c) Gemcitabine.

27. The method of claim 26 wherein said FPT inhibitor is administered twice a day, said Cisplatin is administered once every three or four weeks per cycle, and said Gemcitabine is administered once a week per cycle, said treatment being given for one to seven weeks per cycle.

28. The method of claim 27 wherein said FPT inhibitor is administered in an amount of about 50 mg to about 200 mg twice a day, said Cisplatin is administered once every three or four weeks per cycle in an amount of about 60 to about 100 mg/m², and said Gemcitabine is administered once a week per cycle in an amount of about 750 to about 1250 mg/m².

29. The method of claim 28 wherein said FPT inhibitor is administered in an amount of about 75 mg to about 125 mg twice a day.

30. The method of claim 29 wherein said FPT inhibitor is administered in an amount of about 100 mg twice a day.

31. A method of treating non small cell lung cancer in a patient in need of such treatment comprising administering therapeutically effective amounts of:
(a) an FPT inhibitor of the formula: 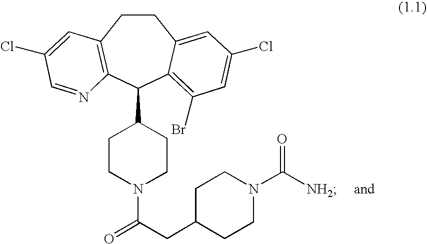
(b) Carboplatin; and
(c) Gemcitabine.

32. The method of claim 31 wherein said FPT inhibitor is administered twice a day, said Carboplatin is administered once every three weeks per cycle, and said Gemcitabine is administered once a week per cycle, said treatment being given for one to seven weeks per cycle.

33. The method of claim 32 wherein said FPT inhibitor is administered in an amount of about 50 mg to about 200 mg twice a day, said Carboplatin is administered once every three weeks per cycle in an amount to provide an AUC of about 5 to about 8, and said Gemcitabine is administered once a week per cycle in an amount of about 750 to about 1250 mg/m².

34. The method of claim 33 wherein said FPT inhibitor is administered in an amount of about 75 mg to about 125 mg twice a day.

35. The method of claim 34 wherein said FPT inhibitor is administered in an amount of about 100 mg twice a day.

36. A method of treating cancer in a patient in need of such treatment comprising administering therapeutically effective amounts of:
(a) an FPT inhibitor of the formula: 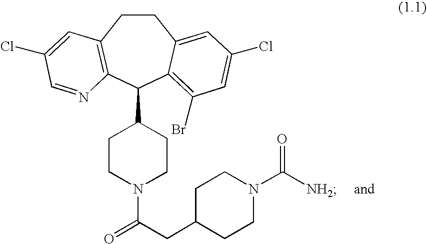
(b) an antineoplastic agent selected from:
(1) EGF inhibitors that are antibodies;
(2) EGF inhibitors that are small molecules;
(3) VEGF inhibitors that are antibodies; or
(4) VEGF kinase inhibitors that are small molecules.

37. The method of claim 36 wherein said antineoplastic agent is selected from: Herceptin, Cetuximab, Tarceva, Iressa, Bevacizumab, IMC-1C-11, SU5416, or SU6688.

38. The method of claim 37 wherein said FPT inhibitor is administered twice a day, said antineoplastic agent that is an antibody is administered once a week per cycle and said antineoplastic agent that is a small molecule is administered daily, said treatment being given for one to four weeks per cycle.

39. The method of claim 38 wherein said FPT inhibitor is administered in an amount of about 50 mg to about 200 mg twice a day, and said antineoplastic agent that is an antibody is administered once a week per cycle in an amount of about 2 to about 10 mg/m², and said antineoplastic agent that is a small molecule is administered in an amount of about 50 to about 2400 mg/m².

40. The method of claim 39 wherein said FPT inhibitor is administered in an amount of about 75 mg to about 125 mg twice a day.

41. The method of claim 40 wherein said FPT inhibitor is administered in an amount of about 100 mg twice a day.

42. The method of claim 2 wherein: said taxane is paclitaxel administered in an amount of about 150 mg to about 300 mg/m² once a week per cycle, and said platinum coordinator compound is carboplatin administered once a week per cycle in an amount to provide an AUC of about 5 to about 8.

43. The method of claim 2 wherein: said taxane is docetaxel administered in an amount of about 50 mg to about 100 mg/m² once a week per cycle, and said platinum coordinator compound is cisplatin administered in amount of about 60 mg to about 100 mg/m² once a week per cycle.

44. The method of claim 1 wherein said cancer being treated is CML and the antneoplastic agents are Gleevec and interferon.

45. The method of claim 1 wherein said cancer being treated is CML and the antneoplastic agents are Gleevec and pegylated interferon.

46. A method of treating AML in a patient in need of such treatment comprising administering therapeutically effective amounts of:
(a) an FPT inhibitor of the formula: 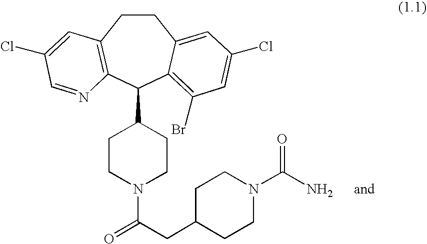
(b) an anti-tumor nucleoside.

47. The method of claim 46 wherein said antinucleoside derivative is Cytarabine.

48. The method of claim 46 further comprising the administration of a therapeutically effective amount of anthracycline.

49. The method of claim 47 further comprising the administration of a therapeutically effective amount of anthracycline.

50. A method of treating non-Hodgkin's lymphoma in a patient in need of such treatment comprising administering therapeutically effective amounts of:
(a) an FPT inhibitor of the formula: 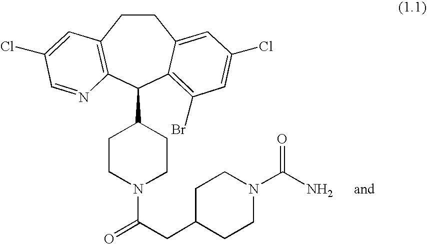
(b) Rituximab.

51. The method of claim 50 further comprising the administration of a therapeutically effective amount of an anti-tumor nucleoside derivative.

52. The method of claim 51 wherein said anti-tumor nucleoside derivative is Fludarabine.

53. A method of treating non-Hodgkin's lymphoma in a patient in need of such treatment comprising administering therapeutically effective amounts of:
(a) an FPT inhibitor of the formula: 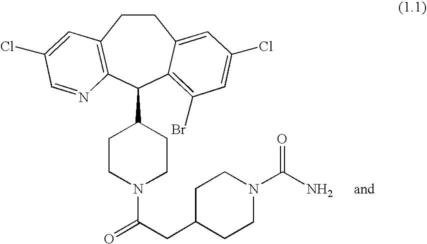
(b) Genasense.

54. A method of treating multiple myeloma in a patient in need of such treatment comprising administering therapeutically effective amounts of:
(a) an FPT inhibitor of the formula: 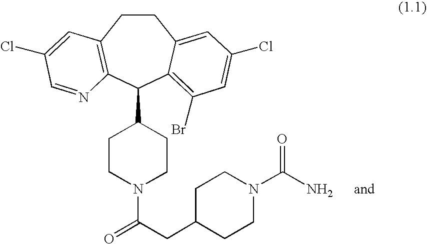
(b) a proteosome inhibitor.

55. A method of treating multiple myeloma in a patient in need of such treatment comprising administering therapeutically effective amounts of:
(a) an FPT inhibitor of the formula: 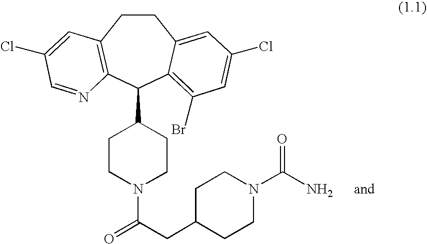
(b) Thalidomide or related imid.

56. The method of claim 55 wherein Thalidomide is administered.

57. The method of claim 44 wherein the FPT inhibitor is administered in an amount of about 50 mg to about 200 mg twice a day.

58. The method of claim 57 wherein said FPT inhibitor is administered in an amount of about 75 mg to about 125 mg twice a day.

59. The method of claim 58 wherein said FPT inhibitor is administered in an amount of about 100 mg twice a day.

60. The method of claim 45 wherein the FPT inhibitor is administered in an amount of about 50 mg to about 200 mg twice a day.

61. The method of claim 60 wherein said FPT inhibitor is administered in an amount of about 75 mg to about 125 mg twice a day.

62. The method of claim 61 wherein said FPT inhibitor is administered in an amount of about 100 mg twice a day.

63. The method of claim 47 wherein the FPT inhibitor is administered in an amount of about 50 mg to about 200 mg twice a day.

64. The method of claim 63 wherein said FPT inhibitor is administered in an amount of about 75 mg to about 125 mg twice a day.

65. The method of claim 64 wherein said FPT inhibitor is administered in an amount of about 100 mg twice a day.

66. The method of claim 49 wherein the FPT inhibitor is administered in an amount of about 50 mg to about 200 mg twice a day.

67. The method of claim 66 wherein said FPT inhibitor is administered in an amount of about 75 mg to about 125 mg twice a day.

68. The method of claim 67 wherein said FPT inhibitor is administered in an amount of about 100 mg twice a day.

69. The method of claim 52 wherein the FPT inhibitor is administered in an amount of about 50 mg to about 200 mg twice a day.

70. The method of claim 69 wherein said FPT inhibitor is administered in an amount of about 75 mg to about 125 mg twice a day.

71. The method of claim 70 wherein said FPT inhibitor is administered in an amount of about 100 mg twice a day.

72. The method of claim 53 wherein the FPT inhibitor is administered in an amount of about 50 mg to about 200 mg twice a day.

73. The method of claim 72 wherein said FPT inhibitor is administered in an amount of about 75 mg to about 125 mg twice a day.

74. The method of claim 73 wherein said FPT inhibitor is administered in an amount of about 100 mg twice a day.

75. The method of claim 54 wherein the FPT inhibitor is administered in an amount of about 50 mg to about 200 mg twice a day.

76. The method of claim 75 wherein said FPT inhibitor is administered in an amount of about 75 mg to about 125 mg twice a day.

77. The method of claim 76 wherein said FPT inhibitor is administered in an amount of about 100 mg twice a day.

78. The method of claim 56 wherein the FPT inhibitor is administered in an amount of about 50 mg to about 200 mg twice a day.

79. The method of claim 78 wherein said FPT inhibitor is administered in an amount of about 75 mg to about 125 mg twice a day.

80. The method of claim 79 wherein said FPT inhibitor is administered in an amount of about 100 mg twice a day.

81. A method of treating squamous cell cancer of the head and neck in a patient in need of such treatment comprising administering therapeutically effective amounts of:
(a) an FPT inhibitor of the formula: 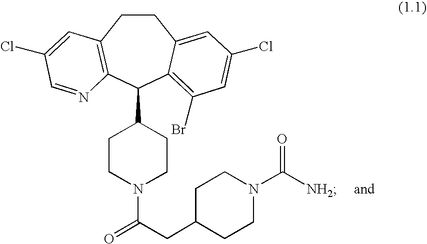
(b) at least two different antineoplastic agents selected from the group consisting of:
(1) taxanes;
(2) platinum coordinator compounds; and
(3) anti-tumor nucleoside derivatives.

82. The method of claim 81 wherein the FPT inhibitor is administered in an amount of about 50 mg to about 200 mg twice a day.

83. The method of claim 82 wherein said FPT inhibitor is administered in an amount of about 75 mg to about 125 mg twice a day.

84. The method of claim 83 wherein said FPT inhibitor is administered in an amount of about 100 mg twice a day.

85. A method of treating non small cell lung cancer in a patient in need of such treatment comprising administering therapeutically effective amounts of:
(a) an FPT inhibitor of the formula: 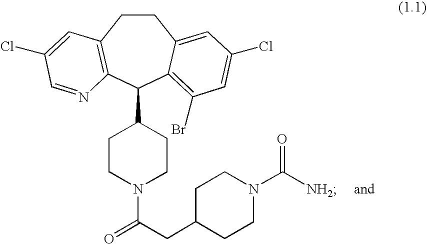
(b) Carboplatin; and
(c) Docetaxel.

86. The method of claim 85 wherein:
(1) said FPT inhibitor is administered in an amount of about 50 mg to about 200 mg twice a day;
(2) said Docetaxel is administered once every three weeks in an amount of about 50 to about 100 mg/m²; and
(3) said Carboplatin is administered once every three weeks in an amount to provide an AUC of about 5 to about 8.

87. The method of claim 86 wherein said docetaxel is administered once every three weeks in an amount of about 75 mg/m² and said Carboplatin is administered once every three weeks in an amount to provide an AUC of about 6.

88. The method of claim 87 wherein said FPT inhibitor is administered in an amount of about 75 mg to about 125 mg administered twice a day.

89. The method of claim 87 wherein said FPT inhibitor is administered in an amount of about 100 mg administered twice a day.
